(19) United States
(12) Patent Application Publication
(10) Pub. No.: US 2008/0060174 A1
(43) Pub. Date: Mar. 13, 2008

(54) Method of Making Closure Tabs with Minimal Waste

(75) Inventors: Carol Erdman, West Chester, PA (US); Michael J. Naughton, Attleboro, MA (US)

Correspondence Address: Covidien, 601 Allendale Road, King of Prussia, PA, 19406, US

(73) Assignee: Tyco Healthcare Retail Services AG, Schaffhausen (CH)

(21) Appl. No.: 11/973,478

(22) Filed: Oct. 9, 2007

Related U.S. Application Data
(62) Division of application No. 11/092,373, filed on Mar. 29, 2005.

(51) Int. Cl. A61F 13/62; A44B 18/00

(52) U.S. Cl. 24/442

(57) ABSTRACT

An absorbent article fastener is provided that includes an absorbent article fastener that is configured for engagement with a landing zone of an absorbent article. The fastener includes an integral substrate having a stretch zone and a shaped deadened zone. A mechanical closure material is mounted with the deadened zone such that the substrate is configured for releasable engagement with the landing zone of the absorbent article. A surface area ratio of the mechanical closure material to the substrate is less than 65%. A method of manufacturing the absorbent article fastener is also provided.

### BACKGROUND

1. Technical Field

The present disclosure generally relates to absorbent articles, and more particularly, to a method of manufacturing closure tabs.

2. Background of the Related Art

Absorbent articles such as, for example, disposable diapers, adult incontinent pads, sanitary napkins, pantiliners, incontinent garments, etc. are generally worn, in cooperation with garments and disposed against a body surface by infants or adult incontinent individuals. The absorbent article is employed to collect and absorb body fluid discharge, such as, for example, blood, menses, urine, aqueous body fluids, mucus and cellular debris. For example, the absorbent article may be disposed between the legs of an individual adjacent a crotch area. The absorbent article is positioned with a garment and drawn into engagement with a body surface of the crotch area to collect fluid discharge.

As is known, absorbent articles typically include a fluid permeable coverstock for engaging the body surface, a fluid impermeable backsheet and an absorbent core supported therebetween. The backsheet serves as a moisture barrier to prevent fluid leakage to the garment. The absorbent core usually includes a liquid retention material that faces the body surface. The absorbent core can include, for example, loosely formed cellulosic fibers, such as, for example, wood pulp, for acquiring and storing fluid discharge.

In addition, fastening of the absorbent articles with an individual requires the use of fasteners or ear members and closure tabs that extend laterally from the body of the absorbent article. In addition, the closure tabs typically include mechanical closure material, for example, hook and/or loop material, adhesive tape or the like. For example, in typical diaper-type garments, the garment is affixed to a wearer by attaching one or more of the closure tabs that extend across the wearer's hips to hold the back and front halves of the garment to one another.

Some of these absorbent articles, however, suffer from manufacturing drawbacks including the amount of scrap or wasted tab material in production thereof.

It would therefore be desirable to overcome the disadvantages and drawbacks of the prior art by providing a more efficiently manufactured closure tab and a corresponding method of making the closure tabs.

### SUMMARY

Accordingly, an efficiently manufactured closure tab and a corresponding method of making the closure tabs with minimal or no waste is disclosed. Objects and advantages of the present disclosure are set forth in part herein and in part will be obvious therefrom, or may be learned by practice of the present disclosure that is realized and attained by the instrumentalities and combinations pointed out in the appended claims for the devices and methods of the present disclosure consisting of its constituent parts, constructions, arrangements, combinations, steps and improvements herein shown and described.

In one particular embodiment, in accordance with the principles of the present disclosure, an absorbent article fastener is provided that includes an absorbent article fastener that is configured for engagement with a landing zone of an absorbent article. The fastener includes an integral substrate having a stretch zone and a shaped deadened zone. A mechanical closure material is mounted with the deadened zone such that the substrate is configured for releasable engagement with the landing zone of the absorbent article. A surface area ratio of the mechanical closure material to the shaped portion of the substrate is less than 65%.

The stretch zone can have a larger surface area dimension relative to the non-stretch zone. The non-stretch zone may be fabricated from a laminate material. The non-stretch zone may be fabricated from a laminate material including a non-woven layer and a non-elastic film layer. The mechanical closure material may be fabricated from a laminate material. The mechanical closure material can include a hook material that engages the loop material of the landing zone of the absorbent article having a hook-to-loop peel force of greater than 400 grams. The mechanical closure material can also include a hook material that engages the loop material of the landing zone of the absorbent article having a hook-to-loop engagement shear force of greater than 4500 grams. The mechanical closure material can include a hook material that engages the loop material of the landing zone of the absorbent article having a hook-to-loop engagement shear-to-peel ratio of less than 15.

In an alternate embodiment, the absorbent article fastener includes an integral substrate including a stretch zone and a shaped non-stretch zone. The stretch zone may include a plurality of spaced apart deadened portions.

In another particular embodiment, in accordance with the principles of the present disclosure, a method of manufacturing a fastener for an absorbent article is provided. The method includes the steps of: providing a fastener material supply including a first stretch zone, a second stretch zone and a non-stretch zone disposed therebetween; mounting mechanical closure material to the non-stretch zone; and cutting the fastener material to form a repeating pattern of substantially identically shaped and oppositely oriented fasteners such that the waste of the fastener material is minimized and each fastener formed has a surface area ratio of the mechanical closure material to the shaped portion of the fastener of less than 65%.

Alternatively, the step of cutting may further include cutting the first stretch zone and the second stretch zone to form rectangular elastic portions of the fasteners. The step of cutting may also include cutting the non-stretch zone to form deadened portions of the fasteners. The step of cutting can also include cutting the fastener material in a sinusoidal pattern. The step of providing may include a non-stretch zone having a polyethylene film joined with the fastener material. The step of providing may also include a fastener material formed of a laminate material.

### BRIEF DESCRIPTION OF THE DRAWINGS

The objects and features of the present disclosure are set forth with particularity in the appended claims. The present disclosure, as to its organization and manner of operation, together with further objectives and advantages may be understood by reference to the following description, taken in connection with the accompanying drawings, in which:

FIG. 1A is a plan view of an absorbent article according to the present disclosure;

FIG. 1B is a partial side view of the absorbent article shown in FIG. 1A;

FIG. 2A is a cutaway plan view of a manufacturing method according to the present disclosure;

FIG. 2B is a side view of the manufacturing method shown in FIG. 2A;

FIG. 3 is an alternate view of the manufacturing method shown in FIG. 2A;

FIG. 4 is a cutaway plan view of an ear portion of the absorbent article shown in FIG. 1 illustrating an alternate embodiment of a fastener; and

FIG. 5 is a cutaway plan view of a method for manufacture of the fastener shown in FIG. 4.

### DETAILED DESCRIPTION OF EXEMPLARY EMBODIMENTS

The exemplary embodiments of the absorbent article and methods of use disclosed are discussed in terms of fluid absorbent articles, and more particularly, in terms of an absorbent article including closure tabs that are manufactured with minimal or no wasted material. The presently disclosed absorbent article is contemplated to be employed with, for example, disposable diapers, training pants, adult incontinent products and other incontinent garments.

In the discussion that follows, the term “body-facing surface” refers to a portion of a structure that is oriented towards a body surface, and the “garment-facing surface” refers to a portion of the structure that is oriented towards a garment and is typically opposing the body-facing surface and may be referred to as such. As used herein, the term “body surface” refers to a portion of an individual's body that the absorbent article is disposed with for collecting and absorbing fluid discharge from the individual. As used herein, the term “absorbent article,” “absorbent garment” or “garment” refers to garments that absorb and contain body waste, and more specifically, refers to garments that are placed against or in proximity to the body of the wearer to absorb and contain the various body discharges and wastes.

A non-exhaustive list of examples of absorbent articles includes diapers, diaper covers, disposable diapers, training pants, feminine hygiene products and adult incontinence products. The term absorbent articles includes all variations of absorbent garments, including disposable absorbent garments that are intended to be discarded or partially discarded after a single use and unitary disposable absorbent garments that have essentially a single structure. As used herein, the term “diaper” refers to an absorbent article generally worn by children and incontinent persons about the lower torso.

The claims are intended to cover all of the foregoing classes of absorbent articles, without limitation, whether disposable, unitary or otherwise. These classifications are used interchangeably throughout the specification, but are not intended to limit the claimed invention. The invention will be understood to encompass, without limitation, all classes of absorbent articles, including those described above.

Absorbent articles and diapers may have a number of different constructions. In each of these constructions it is generally the case that an absorbent core is disposed between a liquid pervious, body-facing topsheet and a liquid impervious, exterior facing backsheet. In some cases, one or both of the topsheet and backsheet may be shaped to form a pant-like article. In other cases, the topsheet, backsheet and absorbent core may be formed as a discrete assembly that is placed on a main chassis layer and the chassis layer is shaped to form a pant-like article. The article may be provided to the consumer in the fully assembled pant-like shape or may be partially pant-like and require the consumer to take the final steps necessary to form the final pant-like shape, such as by fastening one or more fasteners or fastener tabs.

In the case of some diapers and most adult incontinent products, the article often is provided fully formed with factory-made side seams and the article is donned by pulling it up the wearer's legs. In the case of most diapers, wherein, for example, a child lies on his or her back, a caregiver usually places the diaper between the child's legs, pulls the front end of the diaper up between the legs and then attaches one or more closure tabs to the front waist region of the diaper, thereby forming a pant-like structure. For clarity, the present invention is described herein only with reference to a diaper-type article in which the topsheet, backsheet and absorbent core are assembled into a structure that forms a pant-like article when secured on a wearer using fastening devices, although the invention may be used with any other type of absorbent article that may benefit from the use or addition of fastener tabs.

The following discussion includes a description of the absorbent article in accordance with the present disclosure. Reference will now be made in detail to the exemplary embodiments of the disclosure, which are illustrated in the accompanying FIGURES.

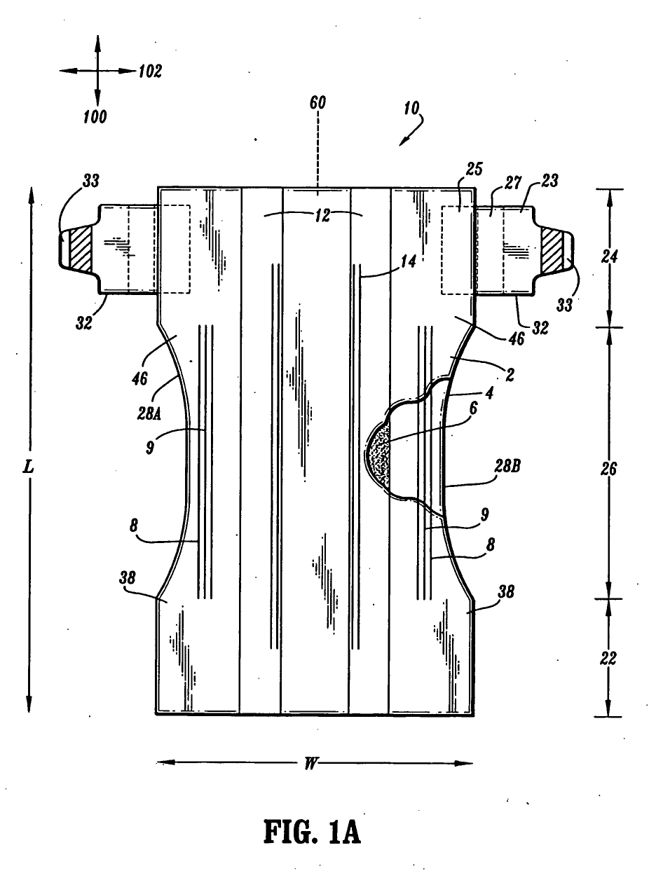

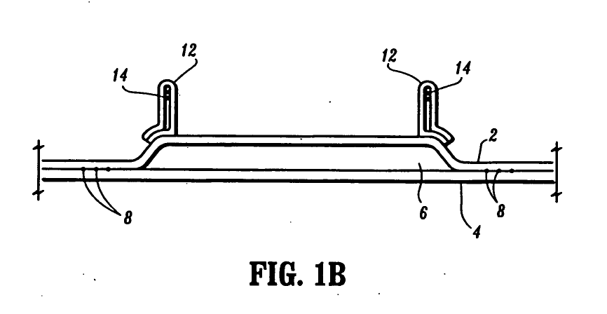

Turning now to the FIGURES, wherein like components are designated by like reference numerals throughout the several views. Referring to FIGS. 1-3, there is illustrated an absorbent article, constructed in accordance with the principles of the present disclosure, such as, for example diaper 10. In FIG. 1A, diaper 10 is shown in a relaxed condition with the effects of the elastics 8, 14 removed for purposes of clarity in the description. The diaper 10 chassis generally has an hourglass shape. The chassis generally can be defined in terms of a front waist region 22, a back waist region 24 and a crotch region 26. Those skilled in the art will recognize that “front” and “back” are relative terms, and these regions may be transposed without departing from the scope of the present invention. Alternatively, the diaper chassis can be configured in a generally rectangular shape or in a “T” shape. The diaper comprises a topsheet 2, a backsheet 4, which may be either a different size than the topsheet 2 or may be substantially coterminous with the topsheet 2 and an absorbent core 6 disposed between at least a portion of the topsheet 2 and backsheet 4. Throughout this description, the terms “topsheet” and “backsheet” denote the relationship of these materials or layers with respect to the absorbent core 6. It is understood that additional layers may be present between or beyond the absorbent core 6 and the topsheet 2 and backsheet 4 and that additional layers and other materials may be present on the side opposite the absorbent core 6 from either the topsheet 2 or the backsheet 4. A pair of leg openings 28A, 28B extend along at least a portion of the crotch region 26 and one or more pairs of leg elastics 8 (three pairs are shown in FIG. 1A) may be disposed to extend adjacent to leg openings 28A, 28B. Of course, in other embodiments, the number of leg elastics 8 may be increased, decreased or omitted altogether.

The diaper 10 generally has a longitudinal direction 100 that extends generally parallel to the front-to-back axis of a wearer, and a lateral direction 102 that extends generally parallel to the side-to-side axis of a wearer. The diaper 10 generally is symmetrical about a longitudinal centerline 60, but also may have asymmetrical components or shapes. The terms “inward” or “proximal,” and “outward” or “distal,” as used herein, refer to positions generally along the lateral direction 102, with “inward” locations being located closer to the longitudinal centerline 60 than “outward” locations.

The diaper may further include a waste containment system in the form of waste containment flaps 12 (also known as inner leg gathers (“ILG's”), unitary leg gathers or standing leg gathers) and one or more pairs of elastics 14. Waste containment flaps 12 extend from the front waist region 22 to the back waist region 24 along opposite sides of the longitudinal center line 60 of the diaper 10 or alternatively only along a portion thereof. The front waist region 22 and rear waist region 24 may include side panels or ear portions 38, 46, extending outward from the leg openings 28a, 28b to provide the garment 10 with an hourglass shape.

A variety of backsheet and topsheet constructions and materials are available and known in the art and the invention is not intended to be limited to any specific materials or constructions of these components. The backsheet 4 may be made from any suitable pliable liquid-impervious material known in the art. Typical backsheet materials include films of polyethylene, polypropylene, polyester, nylon and polyvinyl chloride and blends of these materials. For example, the backsheet can be comprised of a pigmented polyethylene film having a thickness in the range of 0.02-0.04. The moisture-pervious topsheet 2 can be made of any suitable relatively liquid-pervious material known in the art that permits passage of liquid therethrough. Non-woven topsheet materials are exemplary because such materials readily allow the passage of liquids to the underlying absorbent core 6. Examples of suitable topsheet materials include non-woven spunbond or carded webs of polypropylene, polyethylene, nylon, polyester and blends of these materials.

The backsheet 4 and the topsheet 2 may be “associated” with one another. The term “associated” encompasses configurations whereby the topsheet 2 is directly joined to the backsheet 4 by affixing the topsheet 2 directly to the backsheet 4, and configurations whereby the topsheet 2 is indirectly joined to the backsheet 4 by affixing the topsheet 2 to intermediate members which in turn are affixed to the backsheet 4. While the backsheet 4 and topsheet 2 in the present disclosure have substantially the same dimensions, they may also have different dimensions.

In addition, the backsheet 4 may be covered with a fibrous, non-woven fabric layer. Materials for such a fibrous outer liner include a spun-bonded non-woven web of synthetic fibers such as polypropylene, polyethylene or polyester fibers; a non-woven web of cellulosic fibers, textile fibers such as rayon fibers, cotton and the like, or a blend of cellulosic and textile fibers; a spun-bonded non-woven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers.

The backsheet 4 may comprise multiple panels, such as, for example, three panels wherein a central poly backsheet panel is positioned adjacent the absorbent core while distal non-woven breathable side backsheet panels are attached to the side edges of the central poly backsheet panel. The backsheet may also be formed from microporous poly coverstock for added breathability. In other embodiments, the backsheet may be a laminate of several sheets. The backsheet may further be treated to render it hydrophilic or hydrophobic, and may have one or more visual indicators associated with it, such as labels indicating the front or back of the diaper or other characters or colorations. The present invention is not limited to any particular backsheet 4 material or construction.

The topsheet 2 may be formed from one or more panels of material and may comprise a laminated sheet construction. As shown in FIG. 1A, the topsheet 2 comprises one panel across the entire width of the diaper and is formed from a liquid-pervious material that is either hydrophobic or hydrophilic. Topsheet 2 may be made from any number of materials, including synthetic fibers (e.g., polypropylene or polyester fibers), natural fibers (e.g., wood or cellulose), apertured plastic films, reticulated foams and porous foams, etc. Topsheet 2 may be fabricated from a cover stock of single ply non-woven material which may be made of carded fibers, either adhesively or thermally bonded, perforated plastic film, spunbonded fibers, or water entangled fibers, which generally weigh, for example, from 0.3-0.7 oz./yd² and have appropriate and effective machine direction and cross-machine direction strength suitable for use as a baby diaper cover stock material, as are known in the art. Topsheet 2 may extend from substantially the front waist region 22 to the back waist region 24 or a portion thereof, and includes a surfactant (at least along the core area) to aid in the passage of body waste to the core 6.

As shown in FIGS. 1A and 1B, the waste containment flaps 12 may be formed from separate elasticized strips of material that are associated with the topsheet 2, backsheet 4 or both, or otherwise integrated into the garment. The topsheet 2 and backsheet 4 may have similar dimensions or different dimensions, but in either case, the waste containment flaps 12 are attached to the topsheet 2 or to some intermediate element which in turn is attached to the topsheet 2. The waste containment flaps 12 may be treated with a suitable surfactant to modify their hydrophobicity or hydrophilicity or imbued with skin wellness products as desired. Each waste containment flap 12 may include a portion that folds over onto itself to form an enclosure. One or more elastic members 14 may be secured in the enclosure in a stretched condition. Various other configurations of topsheets 2 and waste containment systems, such as flaps 12, are known in the art, and the present invention is not intended to be limited to any particular design for these components.

Each leg opening 28A, 28B may be provided with a leg elastic containment system 9, sometimes referred to as conventional leg gathers. In an illustrative embodiment, three strands of elastic threads 8 are positioned to extend adjacent each leg openings 28A, 28B between the topsheet 2 and the backsheet 4. The selection of appropriate elastics and the construction of leg elastic containment systems are known in the art. For example, the leg elastics 8 may be ultrasonically bonded, heat and pressure sealed using a variety of bonding patterns, or glued to the diaper 10. Leg gathers 9 may be formed with or as separate members from topsheet 2.

Various commercially available materials may be used for the leg elastics 8 and elastic members 14, such as natural rubber, butyl rubber or other synthetic rubber, urethane, elastomeric materials such as spandex, which is marketed under various names, including LYCRA® (DuPont), GLOSPAN™ (Globe) and SYSTEM 7000™ (Fulflex). The present invention is not limited to any particular elastic material or to any particular shape, size or number of elastics.

The underlying structure beneath the topsheet 2 may include, depending on the absorbent article construction, various combinations of elements, but in each embodiment, it is contemplated that the absorbent article will include an absorbent core 6. Although the absorbent core 6 depicted in the FIGURES has a substantially rectangular shape in the plan view, other shapes may be used, such as a “T” shape or an hourglass shape. The absorbent core 6 may extend into either or both of the front and back waist regions 22, 24. The shape and construction of the absorbent core 6 may be selected to provide the greatest absorbency in target areas where body fluids are most likely to strike the diaper 10, which is often referred to as zoned absorbency. The absorbent core 6 may also comprise a number of layers of similar or different construction. The absorbent core may be associated with the topsheet 2, backsheet 4 or any other suitable part of the garment 10 by any method known in the art, in order to fix the absorbent core 6 in place.

Absorbent core 6 may include particles of super absorbent material (SAP) distributed within a fibrous structure. Additional fibrous or particulate additives may be disposed within the absorbent core 6 to add to the core's strength and SAP efficiency or to otherwise enhance the performance of the garment. The absorbent core 6 may be partially or wholly surrounded by additional layers (not shown) added to provide further benefits. The additional layer or layers may comprise any useful layer known in the art or developed hereafter, such as a fluid acquisition layer, a distribution layer, an additional fibrous layer optionally containing super absorbent particles, a wicking layer, a storage layer, or combinations and fragments of these layers. Such layers may be provided to assist with transferring fluids to the absorbent core 6, handling fluid surges, preventing rewet, containing absorbent material, improving core stability or for other purposes.

The absorbent core 6 may be made from any absorbent material or materials, or combinations of such materials, known in the art. The absorbent core 6 may include wood fibers or other fibers such as chemical wood pulp, fibrous absorbent gelling material or any other suitable liquid absorbing material, such as commercially available fluff pulp or fluffed bleached kraft softwood pulp or fibrous absorbent gelling material. Alternatively, the absorbent core 6 may include a combination of a porous fibrous web and super absorbent particles. The absorbent core may be thin to improve the comfort and appearance of a garment.

Diaper 10 is fastened onto a wearer by using fasteners 32. Fasteners 32 are affixed to the chassis of the diaper 10 to extend laterally outward (i.e., in the lateral direction 102) from waist regions 22, 24. Fasteners 32 are positioned to extend outward from the ear portions 46 of the rear waist region 24. However, the fasteners 32 may also be attached to extend outward from the front waist region 22 or from both waist regions 22, 24. The fasteners 32 may extend from one or both lateral sides of the diaper 10.

Fastener 32 are positioned between topsheet 2 and backsheet 4. Each of a pair of closure tabs 33 is attached along distal portion 23 of fastener 32. Closure tab 33 operates by engaging with or adhering to a corresponding surface, landing zone or object (not shown) located on the opposite end of the diaper 10, such as, for example, front waist region 24. Alternatively, fasteners 32 may be attached to any part of the diaper chassis, such as topsheet 2, backsheet 4, outer cover or other layer of the diaper. The fasteners 32 may also be attached to either side of the diaper's chassis, to multiple layers of the chassis, or may be sandwiched between the various sheets or layers comprising the chassis of the diaper 10.

Rectangular fastener 32 has a larger surface area dimension with respect to the smaller closure tab 33. Fastener 32 may be rectangular with sides of equal distance. Fastener 32 may have a “zoned” stretch property, in that, only certain portions or zones thereof will extend or stretch during use. For example, fastener 32 may include center, distal, and proximal sections. Center section 27 is stretchable in lateral direction 102. Distal section 23 is generally not stretchable. Proximal section 25 is generally not stretchable and is attached to the diaper chassis. Fasteners 32 may have deadened zones distal section 23, and proximal section 25 on each side that is generally between 10-30 mm and preferably 20 mm. It is contemplated that the various fastener sections be formed with materials that are elastic, non-elastic or a combination thereof. Fasteners 32 may be attached to the diaper 10 or closure tab 33 or both by adhesive, ultrasonic, thermal bonding or the like.

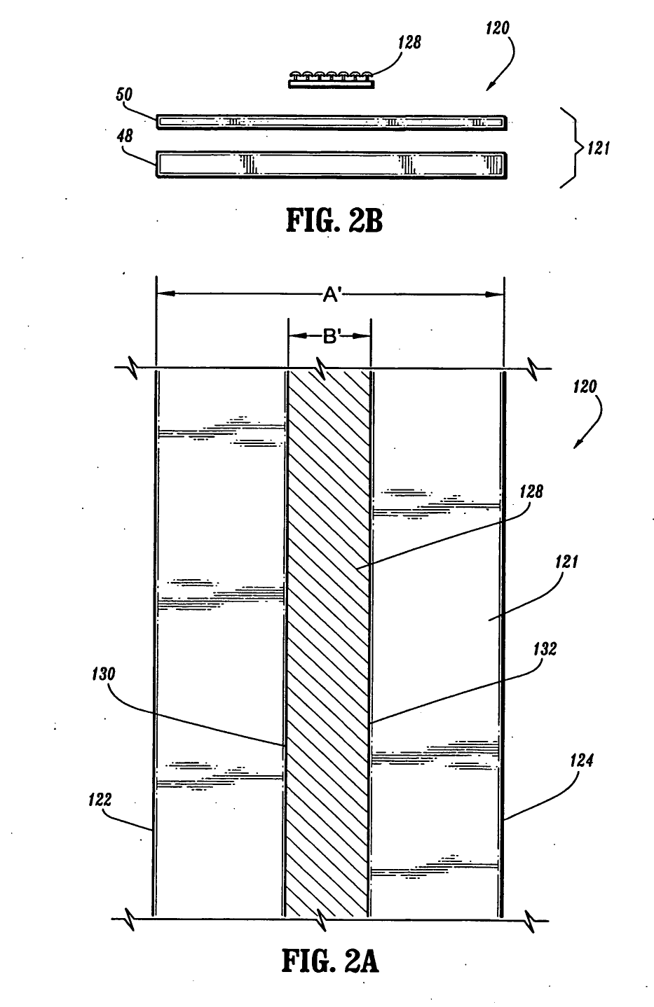

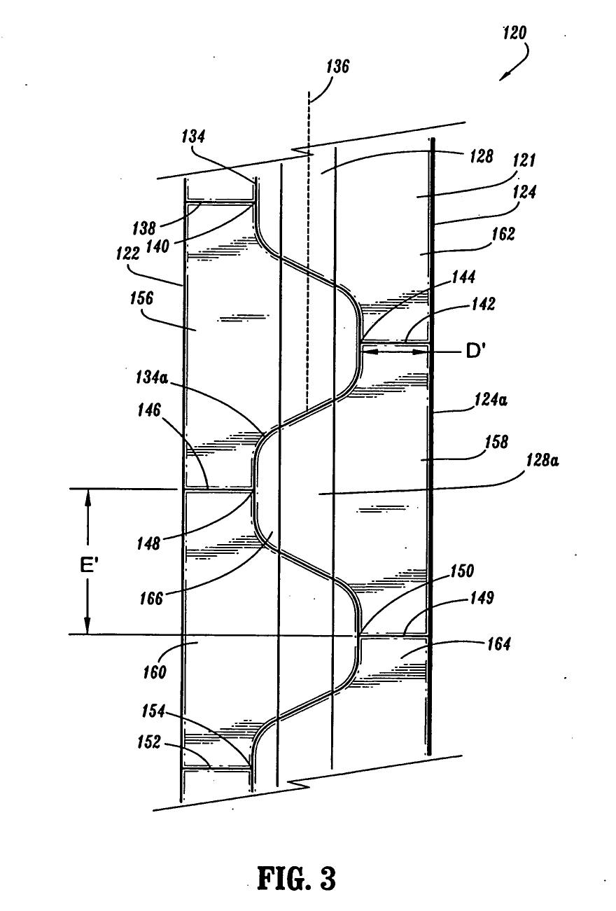

Referring to FIGS. 2A, 2B and 3, a structure and a method are provided for efficiently manufacturing the closure tabs 33 with greatly reduced waste of materials. A strip of tab material 120 is provided for forming a plurality of tabs 33 (FIG. 1A), with minimal or no waste of the material 120. The tab material 120 is cut to form a repeating pattern of substantially identically shaped and oppositely oriented tabs 33. By cutting the material 120 in such a pattern, waste of the material 120 between and surrounding the tabs 33 is decreased or preferably eliminated.

The material 120 from which tabs 33 are to be formed includes a first substrate, such as, for example, a non-stretch material 121. However, it is also contemplated that the tab material 120 may include elastic material, or a combination of stretch and non-stretch materials. The tab material 120 also includes a second substrate, such as, for example, a mechanical closure material 128. As part of the tab 33 when formed, the mechanical closure material 128 engages a corresponding surface on the region of the diaper 10 opposite that to which the tab 33 is attached. The mechanical closure material 128 may comprise any hook-and-loop type fastener, adhesive fastener, or other type of fastener that is capable of holding diaper 10 on a wearer.

The mechanical closure material 128 has formed thereon a plurality of hook stems or hooks and is herein referred to as hook material 128. In this embodiment, the non-stretch material 121 extends laterally a distance A′ between a first edge 122, and a second edge 124. The mechanical closure material 128 extends longitudinally along the center of the non-stretch material 121. The mechanical closure material 128 spans laterally a distance B′ between left edge 130 and right edge 132.

It is contemplated that either of the hook material 128 or the non-stretch material 121 may comprise a laminate of two or more layers or materials. The non-stretch material 121 may be a white non-stretch bi-laminate such as a Tredegar 100D available from Tredegar Film Products of Richmond, Va. As can be seen, the non-stretch material 121 may include a non-woven layer 48 adjacent a layer of non-stretch or non-elastic film 50. The non-woven layer 48 may be spunbonded polypropylene, polyethylene, or other non-woven material, such as, for example, a white spunbonded material weighing generally between 10-45 gsm and preferably 25 gsm. Suitable hook-and-loop fasteners are readily available from 3M Corp. (St. Paul, Minn.), Aplix, Inc. (Charlotte, N.C.), and Velcro USA, Inc. (Manchester, N.H.).

The hook material 128 may be, for example, a white hook element with a pin density generally between 1200-1600 and preferably 1400, such as item number CS600 available from 3M Corporation. The bonding between layers 48, 50, 128 of the tab material 120 may be accomplished using joining methods known in the art, such as, for example, compression bonds, heat bonds, ultrasonic bonds, adhesives and the like, or combinations of different bonding methods. For example, the hook material 128 may be adhered to the non stretch material 121 using an adhesive such as an NS 34-5640 as available from National Starch Corporation. In any case, the construction of such laminates is known in the art, and a skilled artisan will be able to provide a suitable laminate or other design for the tab material 120 without undue experimentation.

The tab material 120, comprised of the non-stretch material 121 and the hook material 128, is divided or cut along a plurality of tab-forming lines to produce a plurality of substantially identical and oppositely oriented tabs 156, 158, 160, as shown in FIG. 3. The tab material 120 is cut in an alternating or periodic pattern, such as, for example, a sinusoidal 134 which cycles about a longitudinal centerline 136 of the tab material 120. Centerline 136 also bisects the hook material 128 longitudinally. Tab material 120 may be cut in alternate shapes, such as, for example, arcuate shapes, polygonal, etc.

During a first cycle, the sinusoid 134 starts at an amplitude point 140. From the point 140, the sinusoid line 134 traverses the hook material 128 and intersects a point 144 on the left side of the non-stretch material 121 near an apex of the sine wave. From the point 144, the sinusoid 134 traverses the hook material 128 and intersects an apex point 148 opposite the point 144, completing the first cycle. The sinusoid 134 thereafter continues along the tab material 120 in a repeating pattern.

Tab-forming cuts are also made in the tab material 120 along a plurality of lateral lines. The lateral cuts or lines each extend a distance D′ and are spaced longitudinally an equal distance E′ from each other. For example, a first line 138 extends from the left edge 122 of the non-stretch material 121 to the point 140. A second line 142 is disposed frontward from the first line 138, and extends from the right edge 124 of the non-stretch material 121 to the point 144. A third line 146 extends from the left edge 122 of the tab material 120 to the point 148. A fourth cut 149 extends from the right edge 124 of the tab material to a point 150. A fifth cut 152 extends from the left edge 122 of the tab material 120 to a point 154.

Cutting the tab material 120 forms identically shaped and oppositely oriented tabs, such as, for example, a first tab 156, a second tab 158, and third tab 160, without waste of the tab material 120. For purposes of example, the tab 158 can be oriented for attachment to the left diaper fastener 32 shown in FIG. 1A. The line 142 cut in the tab material 120 delineates or defines the rear edge of the tab 158. The line 142 also defines the front edge of a tab 162 (shown partially) adjacent the tab 158. The line 149 cut in the tab material 120 defines the front edge of tab 158, and also defines a rear edge of an adjacent tab 164 (shown partially). A portion of the right edge 124 of the tab material 120 defines a proximal edge 124a of the tab 158. The proximal edge 124 extends a distance 2E′ and may be attached to the distal end of the fastener 32 shown in FIG. 1A. A curved or distal edge 134a of the tab 158 is defined by a portion of the line 134 that cycles for one period from the point 144 to the point 150. This line portion 134a also defines in part the respective curved edges of the adjacent tabs 156 and 160. The tab 158 includes hook material 128a for fastening a diaper and which is bound longitudinally by portions of the curved edge 134a. A lifting region 166 for lifting or actuating the tab 158 is disposed at or near the distal edge 134a of tab 158. The lifting region 166 includes the non-stretch material 121 and may not include the hook material 128a.

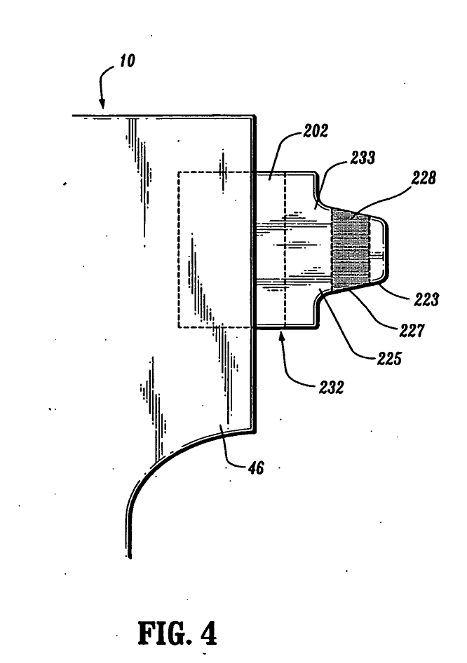

Referring to FIG. 4, an alternate embodiment of diaper 10, similar to that described with regard to FIGS. 1A and 1B, is fastened onto a wearer by employing fasteners 232. Fasteners 232 are affixed to the chassis of diaper 10 to extend laterally outward from waist regions 22, 24 (shown in FIG. 1A). Fasteners 232 are mounted to ear portions 46 and positioned to extend outward from ear portions 46. Fasteners 232 may also be attached to extend outward from front waist region 22 or from both waist regions 22, 24. Fasteners 232 may extend from one or both lateral sides of diaper 10.

Fasteners 232 are affixed to backsheet 4 (shown in FIG. 1B). Alternatively, fasteners 232 may be positioned between topsheet 2 and backsheet 4. Fasteners 232 include a tab portion 233 that extends distally from a rectangular portion 202 of fastener 232. Tab portion 233 and rectangular portion 202 form an integral substrate of fastener 232. Tab portion 233 is configured to engage with a corresponding surface, landing zone or object (not shown) located on the opposite end of diaper 10, such as, for example, front waist region 24. Alternatively, fasteners 232 may be attached to any part of the diaper chassis, such as topsheet 2, backsheet 4, outer cover or other layer of the diaper. Fasteners 232 may also be attached to either side of the diaper's chassis, to multiple layers of the chassis, or may be disposed between the various sheets or layers comprising the chassis of diaper 10. It is contemplated that fasteners 232 may be monolithically formed, or alternatively, tab portion 233 and rectangular portion 202 are separately attachable by various joining methods as described herein.

Fasteners 232 are substantially formed of an elastic stretch material, with deadened non-stretch zones, as will be discussed. Rectangular portion 202 has a larger surface area dimension with respect to the smaller tab portion 233 and has sides of equal distance. It is contemplated that fasteners 232 may be variously configured and dimensioned, whereby the dimensions of the sides of rectangular portion 202 may be varied, or alternatively, portions 202, 233 may be similar in size, or alternatively, tab portion 233 may be larger than rectangular portion 202.

Fasteners 232 have a “zoned” stretch property in that only portions or zones thereof will extend or stretch during use. For example, rectangular portion 202 is stretchable in the lateral direction. Tab portion 233 has a non-stretch property and includes a deadened zone defined by a proximal section 225, central section 227 and distal section 223. Proximal section 225 includes non-stretch material and extends from rectangular portion 202. Distal section 223 and central section 227 also include non-stretch material. Tab portion 233 has a layer of non-stretch material adhered thereto to form a deadened zone of fastener 232. It is contemplated that tab portion 233 may include a layer of stretch material that is monolithically formed with rectangular portion 202 whereby the layer of non-stretch material is adhered thereto. Alternatively, tab portion 233 may be formed of non-stretch material and joined with rectangular portion 202. It is further contemplated that tab portion 233 may include non-deadened zones or zones of stretch material. It is envisioned that rectangular portion 202 may include deadened zones.

Central section 227 is configured for attachment with mechanical closure material 228 as will be discussed. This advantageous configuration eliminates the need for additional layers, substrates, etc. to facilitate mounting of mechanical closure material 228 with fasteners 232. It is contemplated that the various fastener sections may be formed with materials that are elastic, non-elastic or a combination thereof. Fasteners 232 may be attached to diaper 10 by adhesive, ultrasonic, thermal bonding or joining methods discussed herein.

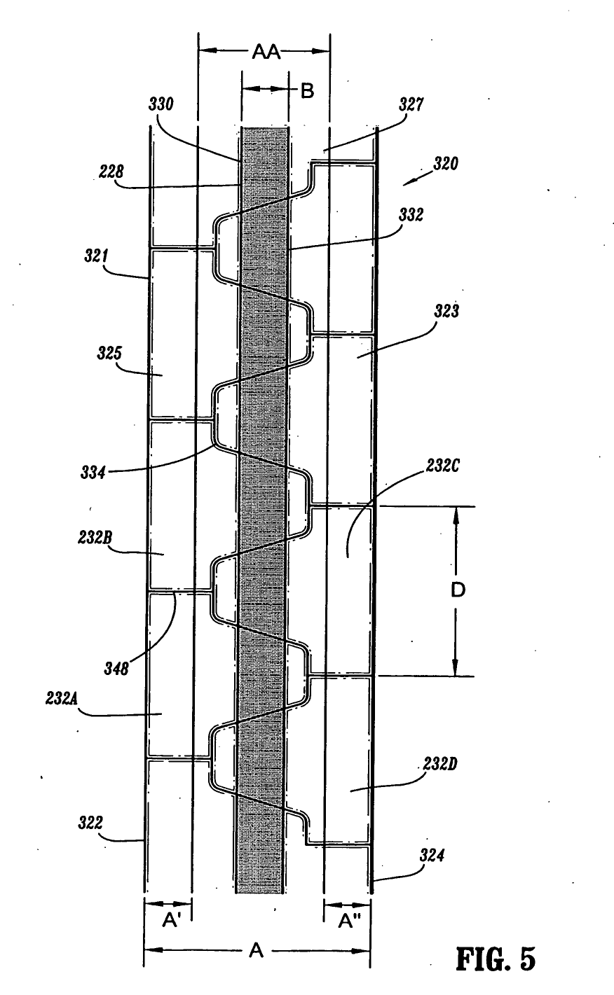

Referring to FIG. 5, a structure and a method are provided for efficiently manufacturing fasteners 232 with greatly reduced waste of materials. An elongated strip supply of fastener material 320 is provided for forming a plurality of fasteners 232 (FIG. 4), with minimal or no waste of material 320. Fastener material 320 is cut to form a repeating pattern of substantially identically shaped and oppositely oriented fasteners 232. By cutting material 320 in such a pattern, waste of material 320 between and surrounding fasteners 232 is decreased or eliminated.

Material 320 from which fasteners 232 are to be formed includes a first substrate, such as, for example, a zoned stretch material 321. It is contemplated that fastener material 320 may include only elastic material, only non-stretch material or combinations thereof. Fastener material 320 also includes a second substrate, such as, for example, a non-elastic material that is disposed with stretch material 321 to form a deadened zone 327. A third substrate, such as, for example, a mechanical closure material 228 (similar to mechanical closure material 128 shown in FIG. 2B) or hook material 228, similar to that described, is also disposed with stretch material 321. The substrates of material 320 may be disposed in an end to end, or edge to edge orientation to form a monolithic or integrally joined material 320. Alternatively, the substrates of material 320 may be layered at various degrees of overlap, ranging from edge to edge to complete overlap.

Zoned stretch material 321 extends laterally a distance A between a first edge 322 and a second edge 324. Material 321 has a first stretch zone 325 formed of elastic material and extending a distance A′ from first edge 322. A second stretch zone 323 is also formed of elastic material and extends a distance A″ from second edge 324. It is contemplated that the stretch zones may be variously dimensioned. The non-stretch material, which forms deadened zone 327, is joined with the stretch material to form fastener 232. The non-stretch material may be joined with stretch material 321 by various joining methods discussed herein. Deadened zone 327 extends a distance AA. It is envisioned that zoned stretch material 321 may include one or a plurality of deadened zones. It is further envisioned that deadened zone 327 may be variously oriented with zoned stretch material 321.

Zones 325, 327 and 323 of material 321 facilitate manufacture of fasteners 232 and formation of rectangular portion 202, proximal section 225, central section 227 and distal section 223 of tab portion 233. For example, to form a repeating pattern of identically shaped fasteners 232A and 232B, first stretch zone 325 provides elastic stretch material that forms rectangular portion 202. Deadened zone 327 provides a non-elastic, non-stretch area that forms proximal section 225, central section 227 and distal section 223 of tab portion 233.

To form identically shaped and oppositely oriented fasteners 232C and 232D, second stretch zone 323 provides elastic stretch material that forms rectangular portion 202. Deadened zone 327 provides a non-elastic, non-stretch area that forms proximal section 225, central section 227 and distal section 223 of tab portion 233.

Hook material 228 is adhered to deadened zone 327 by methods similar to those described above. Hook material 228 extends longitudinally along the center of the zoned-stretch material 321. Hook material 228 spans laterally a distance B between a first edge 330 and a second edge 332 of deadened zone 327. It is envisioned that hook material 228 engages with a corresponding loop landing zone of diaper 10 having a hook to loop peel force of greater than 400 grams, although according to the particular application, other ranges are envisioned. It is further envisioned that hook material 228 engages the loop landing zone having a hook-to-loop engagement shear force of greater than 4500 grams, although other ranges are contemplated. It is further envisioned that hook material 228 engages the loop landing zone having a hook-to-loop engagement shear to peel ratio of less than 15, although other ratios are contemplated. For example, a hook-to-loop engagement shear force of 4600 grams divided by a hook-to-loop peel force of 410 grams results in a shear-to-peel ratio of 11.2.

It is contemplated that zoned-stretch material 321 may comprise a laminate of two or more layers or materials. It is further contemplated that deadened zone 327 is a polyethylene film inserted between layers of stretch material 321. Alternatively, the film can be adhered to an outer surface of stretch material 321. Zoned stretch material 321 may include a non-woven layer or a non-elastic film. The non-woven layer may be spunbonded polypropylene, polyethylene, or other non-woven material. It is contemplated that deadened zone 327 may have various width dimensions according to the particular application, including a range of 30-50 mm, although other ranges are envisioned. It is further contemplated that the surface area ratio of hook material 228 to the shaped portion of fastener 232 is less than 65%, although according to a particular application, other ranges are envisioned.

Fastener material 320, including zoned stretch material 321, deadened zone 327 and hook material 228, is divided or cut along a plurality of fastener-forming lines to produce a plurality of substantially identical and oppositely oriented fasteners, such as, for example, 232A, 232B, 232C, 232D. Fastener material 320 is cut in an alternating or periodic shaped pattern, such as, for example, a sinusoidal 334, similar to that described, for traversing zones 325, 327 and 323 of material 321 in a repeating pattern to form fasteners 232. It is contemplated that fastener material 320 may be cut in alternate shapes, such as, for example, arcuate shapes, polygonal, etc.

Fastener-forming cuts 348 are also made laterally in material 321 to form fasteners 232. Lateral cuts 348 define the fastener cut length that extends to a distance D. It is contemplated that dimension D may be variously sized according to the application, including 60 mm, although other ranges are envisioned.

Cutting fastener material 320 forms identically shaped and oppositely oriented fasteners 232, such as, for example, fasteners 232A, 232B, 232C and 232D, without waste of fastener material 320. For purposes of example, fastener 232A can be oriented for attachment to ear portion 46 of diaper 10 shown in FIG. 4.

The invention of the present disclosure may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. An absorbent article fastener configured for engagement with a landing zone of an absorbent article, the fastener comprising:
an integral substrate including a stretch zone and a shaped deadened zone; and
a mechanical closure material being mounted with the deadened zone such that the substrate is configured for releasable engagement with the landing zone of the absorbent article, wherein a surface area ratio of the mechanical closure material to the shaped deadened zone is less than 65%.

2. An absorbent article fastener as recited in claim 1, wherein the stretch zone has a larger surface area dimension relative to the shaped deadened zone.

3. An absorbent article fastener as recited in claim 1, wherein the shaped deadened zone is fabricated from a laminate material.

4. An absorbent article fastener as recited in claim 1, wherein the shaped deadened zone is fabricated from a laminate material including a non-woven layer and a non-elastic film layer.

5. An absorbent article fastener as recited in claim 1, wherein the mechanical closure material is fabricated from a laminate material.

6. An absorbent article fastener as recited in claim 1, wherein the mechanical closure material includes a hook material that engages a loop material of the landing zone of the absorbent article having a hook-to-loop peel force of greater than 400 grams.

7. An absorbent article fastener as recited in claim 1, wherein the mechanical closure material includes a hook material that engages a loop material of the landing zone of the absorbent article having a hook-to-loop engagement shear force of greater than 4500 grams.

8. An absorbent article fastener as recited in claim 1, wherein the mechanical closure material includes a hook material that engages a loop material of the landing zone of the absorbent article having a hook-to-loop engagement shear-to-peel ratio of less than 15.

9. An absorbent article fastener comprising:
an integral substrate including a stretch zone and a shaped non-stretch zone; and
a mechanical closure material being mounted with the non-stretch zone, wherein a surface area ratio of mechanical closure material to the shaped non-stretch zone is less than 65%.

10. An absorbent article fastener as recited in claim 9, wherein the stretch zone has a larger surface area dimension relative to the non-stretch zone.

11. An absorbent article fastener as recited in claim 9, wherein the stretch zone includes a deadened portion.

12. An absorbent article fastener as recited in claim 9, wherein the stretch zone includes a plurality of spaced apart deadened portions.

13. An absorbent article fastener as recited in claim 9, wherein the non-stretch zone is fabricated from a laminate material.

14. An absorbent article fastener as recited in claim 9, wherein the non-stretch zone is fabricated from a laminate material including a non-woven layer and a non-elastic film layer.

15. An absorbent article fastener as recited in claim 9, wherein the mechanical closure material is fabricated from a laminate material.

16. A method of manufacturing a fastener for an absorbent article, the method comprising the steps of:
providing a fastener material supply including a first stretch zone, a second stretch zone and a non-stretch zone disposed therebetween;
mounting mechanical closure material to the non-stretch zone; and
cutting the fastener material to form a repeating pattern of substantially identically shaped and oppositely oriented fasteners such that the waste of the fastener material is minimized and each fastener formed has a surface area ratio of the mechanical closure material to the non-stretch zone of the fastener of less than 65%.

17. A method of manufacturing a fastener for an absorbent article as recited in claim 16, wherein the step of cutting further includes cutting the first stretch zone and the second stretch zone to form rectangular elastic portions of the fasteners.

18. A method of manufacturing a fastener for an absorbent article as recited in claim 16, wherein the step of cutting further includes cutting the non-stretch zone to form deadened portions of the fasteners.

19. A method of manufacturing a fastener for an absorbent article as recited in claim 16, wherein the step of cutting includes cutting the fastener material in a sinusoidal pattern.

20. A method of manufacturing a fastener for an absorbent article as recited in claim 16, wherein the step of providing includes a non-stretch zone having a polyethylene film joined with the fastener material.

21. A method of manufacturing a fastener for an absorbent article as recited in claim 16, wherein the step of providing includes a fastener material formed of a laminate material.
